(19) United States
(12) Patent Application Publication
(10) Pub. No.: US 2015/0192554 A1
(43) Pub. Date: Jul. 9, 2015

(54) Gas sensor and method for analyzing the measured variables

(71) Applicant: Robert Bosch GmbH, Stuttgart (DE)

(72) Inventors: Paul KOOP, Stuttgart (DE); Frank Schaefer, Tuebingen (DE)

(21) Appl. No.: 14/592,151

(22) Filed: Jan. 8, 2015

(30) Foreign Application Priority Data
Jan. 8, 2014 (DE) 10 2014 200 132.3

(51) Int. Cl. G01N 33/00; G08B 21/14

(52) U.S. Cl. CPC G01N 33/007; G01N 33/004; G08B 21/14

(57) ABSTRACT

A method and an analysis unit for analyzing the measured variables of a gas sensor, and a gas sensor which is operated using this method, provides that the fundamental method detects measured variables of a gas sensor, which represent the concentration of the gas to be monitored. Subsequently, two analysis modes are provided in the method, which differ essentially due to the speed and precision of the measured variable analysis. In a first analysis mode, at least one measured variable and/or the difference between two successive measured variables is compared to a threshold value. Depending on the output of the comparison, subsequently a first gas concentration signal is generated or not. The situation is similar in the second analysis mode, an average value calculation of at least two measured variables being carried out here and a second gas concentration signal being generated as a function of the result.

### RELATED APPLICATION INFORMATION

The present application claims priority to and the benefit of German patent application no. 10 2014 200 132.3, which was filed in Germany on Jan. 8, 2014, the disclosure of which is incorporated herein by reference.

### FIELD OF THE INVENTION

The present invention relates to a method and an analysis unit for analyzing the measured variables of a gas sensor, and a gas sensor which has such an analysis unit.

### BACKGROUND INFORMATION

If health-hazardous operating materials are used in air conditioners, a health-hazardous situation may result for those present if the operating materials escape into enclosed spaces. To moderate or prevent these critical situations, gas sensors may be used, which output warnings or initiate countermeasures if corresponding gas concentrations are achieved.

In addition, however, the increase of a gas concentration to a level below a health-hazardous situation may also result in losses of comfort. Thus, for example, under certain circumstances, the interior in a vehicle may collect a sufficient amount of CO₂ during operation of the air conditioner in recirculated air operation to impair the concentration capability. In contrast to the above-described detection of a leak of the air conditioner, in the case of which a rapid increase of the CO₂ proportion occurs, a slower increase is to be detected in this case.

An air conditioner having a gas sensor is discussed in patent document DE 10 2006 044 083 A1, which may be operated in multiple operating modes. Thus, a more active operating mode may be provided, in which both a possible leak of the refrigerant of the air conditioner and also a concentration increase of CO₂ due to the respiration air of the occupants may be monitored. A similar system is discussed in patent document DE 103 18 504 A1.

### SUMMARY OF THE INVENTION

An object of the present invention is to provide an analysis method, using which both the rapid increase and also the slow increase of a gas concentration, for example, CO₂, may be detected. The requirements for the absolute precision with respect to the detection of both concentrations may be defined differently.

In the present invention, a method and an analysis unit for analyzing the measured variables of a gas sensor and a gas sensor which is operated using this method are described. For this purpose, it is provided that the fundamental method detects measured variables of a gas sensor, which represent the concentration of the gas to be monitored. Subsequently, two analysis modes are provided in the method, which differ essentially due to the speed and precision of the measured variable analysis. In a first analysis mode, at least one measured variable and/or the difference between two successive measured variables is/are compared to a threshold value. Depending on the output of the comparison, a first gas concentration signal is subsequently generated or not. The situation is similar in the second analysis mode, an average value calculation of at least two measured variables being carried out here or another filter algorithm being used and a second gas concentration signal being generated as a function of the result. The required components for the analysis logic and sensor calibration, for example, a microcontroller or memory, may be implemented both in the sensor and also in the external analysis unit, for example, an ECU.

Using such a two-part analysis of the measured values of a gas sensor, both a faster increase and also a slower increase of the concentration of a specific gas component may be reliably detected.

In one refinement of the present invention, in the first analysis mode, if the first threshold value is exceeded by the one measured variable or the difference of two successive measured variables, it is recognized whether a safety-critical or health-hazardous situation is present. The first gas concentration signal is only generated upon recognition of this situation. This first gas concentration signal may optionally be used both as a warning and also as a control signal for initiating countermeasures, for example, the automatic opening of windows.

In the second analysis mode, a rapid threshold value query may also be carried out. However, since averaging over the measured variables takes place in this mode for comfort purposes, only a slow but possibly continuous increase of the gas concentration may be recognized. A second threshold value is therefore provided, which indicates a comfort-critical situation if it is exceeded by the mean value. The second gas concentration signal may also be used in this mode for a warning message or as a control signal to initiate measures with respect to comfort improvement. It is conceivable in this case that the air conditioner switches over from recirculating air operation to fresh air operation, to reduce the CO₂ concentration.

An embodiment of the present invention is particularly advantageous, in which a gas sensor is used, which has at least two detector channels. In this case, the measured variables or measured values from the first detector channel could be used essentially exclusively for the first analysis mode or for deriving the first gas concentration signal. In contrast, the measured variables or measured values of the second detector channel could be used essentially exclusively for the second analysis mode or for deriving the second gas concentration signal. In this case, it would even be conceivable that balancing of the two measured variables, which are detected separately from one another, in the detection channels would be possible. Optionally, however, an additional reference channel of the gas sensor may also be used for the calibration, for example, in that the measured variables or measured values of one detection channel are modified if aging/drift or soiling is recognized.

Further advantages result from the following description of exemplary embodiments or from the further descriptions herein.

### BRIEF DESCRIPTION OF THE DRAWINGS

FIG. 1 schematically shows one possible implementation of the claimed present invention on the basis of a block diagram.

FIG. 2 shows one specific embodiment of an analysis method of the present invention.

### DETAILED DESCRIPTION

As explained at the outset, gas sensors and in particular CO₂ sensors fulfill different functions in the vehicle. If a CO₂ sensor is used in conjunction with an air conditioner operated using a refrigerant based on CO₂, the gas sensor may therefore be used both for leak monitoring and also for comfort purposes.

For the function of a safety sensor, the gas sensor must have a short response time, to be able to recognize a rapid hazardous increase of the CO₂ in the passenger compartment. The reaction times are typically <0.5 seconds here. Since higher concentrations, for example, in the range of 0.5% to 3%, are achieved rapidly in the event of a leak, absolute precision of the measurement is not necessary. Rather, it must be recognized whether a critical and therefore hazardous threshold for the occupants of the vehicle is exceeded or whether the speed of increase is so large that a leak may be presumed.

If the gas sensor is used for comfort purposes, in contrast, a higher requirement is provided for the absolute precision at lower CO₂ concentrations (for example, 0.04% to 0.1%). To achieve this precision, a longer measurement is typically necessary, for example, over one second or longer.

The gas sensors or CO₂ sensors used are typically each optimized to one of the two usage cases, since the orientation to speed and precision require partially contrary measuring devices and analyses. However, a device and a method are to be described by the present invention, using which both applications may be sufficiently covered.

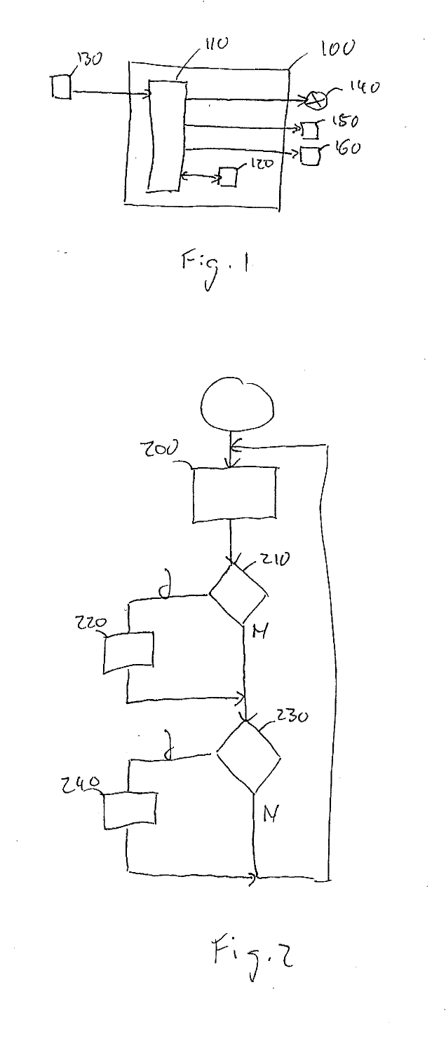

FIG. 1 schematically shows one exemplary embodiment of the present invention for implementing an analysis unit 100 of a gas sensor 130. This gas sensor 130 is described hereafter as a CO₂ sensor, but may also detect the concentrations of any other gas. Analysis unit 100 may also process the corresponding detected measured variables of such a gas sensor. In a first embodiment, CO₂ gas sensor 130 only has one detector channel and optionally one reference channel. The measured variables of this detector channel are input into an analysis arrangement or a processing unit 110. Proceeding from these measured variables, a two-stage comparison of the measured variables or the value derived from these measured variables to one or more threshold values, which are stored in a memory 120, for example, is carried out in processing unit 110. These threshold values may either be provided unchanged or may be adapted by processing unit 110 or by an external intervention. As a function of the comparison, i.e., if it is established that a threshold value is exceeded in the first and/or second stage, an acoustic and/or optical message 140 may be output to the vehicle occupants. Alternatively or optionally additionally, a first measure 150 may be initiated upon recognition of a leak in the first stage, to counteract the hazardous situation of the excessively high CO₂ component or the CO₂ increase. This could be achieved, for example, by automatically lowering the windows. If it is established that a second threshold value is exceeded in the second stage, a second measure may be carried out in the scope of the comfort function, i.e., switching over from recirculated air operation to a supply of air from the outside.

One possible analysis method, which may be carried out in a processing unit 110, for example, is shown in FIG. 2 on the basis of a flow chart. After the start of the method, the measured variable of the (CO₂) gas sensor is detected in a first step 200. Subsequently, in the scope of the first stage, it is checked in step 210 whether this measured variable exceeds a first threshold value SW1, which indicates that the gas concentration has assumed an excessively high value. For the case of CO₂ monitoring, a first threshold value of 0.5% to 3% may be used in this case. Additionally or alternatively, it may also be checked whether the increase of the gas concentration is critical by comparing the difference between two successive measured variables to a corresponding further first threshold value. If it is established in at least one of the cases that this threshold value is exceeded, in a step 220, a warning is output and/or a corresponding first measure 150 is initiated. Optionally, the method may also be ended or restarted in this case. Alternatively, however, the sequence may also continue with further step 230, which is provided for the case that it has not been established that first threshold value SW1 is exceeded. This step 230 represents the second stage of the gas monitoring. In this step 230, an average value calculation of the detected measured variables is carried out and the average value thus obtained is compared to a second threshold value SW2, which is lower than first threshold value SW1. If the average value exceeds a second threshold value SW2, which typically has a value of 0.04% to 0.1%, second measure 160 is initiated in step 240. The method may subsequently be ended, restarted, or carried out again at step 200. However, if it is not established in step 230 that the threshold value is exceeded, renewed detection of a measured variable is carried out, which is input into the two-stage analysis.

Overall, it is provided that this two-stage comparison process is carried out sufficiently rapidly that a sufficiently rapid measured variable detection is provided for the first comparison. In contrast, in step 230 a comparatively long average value calculation is carried out, which is more precise the more measured variables are input into the calculation. Typical values for the measured value detection are, for example, in step 200, approximately 30 ms and 1 second for the average value calculation.

Alternatively, it may also be provided in another exemplary embodiment that the two stages run in parallel independently of one another in processing unit 110 and the detected measured variables may be analyzed independently of one another. In this case, it may also be provided as in the preceding exemplary embodiment that the measured variables and/or mean values are stored for the comparison in memory 120, optionally with a chronological decay constant. Due to the parallel processing of the measured variables, rapid recognition and also a chronologically slower average value calculation may thus be achieved, with the aid of which the particular measure may be initiated if the corresponding threshold value is exceeded.

In general, the use of two independent detector channels enables balancing of the detected measured variables by way of an additional measured value detection. Both solely a comparison and also a correction of the measured valuables are possible. It is thus conceivable that the measured variables of the reference channel are used as calibration values for the actual measurement channel. Furthermore, it is possible that the reference channel detects at a wavelength which is not influenced by the gas to be studied (and ideally also not by other gases). Aging and drift effects within the optical beam path (radiation source, reflector) may thus be calculated out. In that processing unit 110 thus also detects the measured variables of the reference channel, it may be recognized whether the threshold values are actually exceeded by the measured variables. The measured variables may optionally also be adapted to the variables detected by the reference channel or corrected.

Furthermore, it may be provided that the gas sensor has two or more separate detector channels and one reference channel. In this case, it is possible to have the comparison be carried out in the first stage by way of first measured variables from a first, faster detector channel and the comparison in the second stage be carried out by way of second measured variables from a second, more precise detector channel. In this case, both a separate analysis of the two measured variables in two different analysis methods or also a shared two-stage analysis method according to FIG. 2 are possible.

Optionally, it is also possible to compare the measured variables of the first and the second detector channels. It could thus be ascertained how the difference of the present change due to the rapid measurement changes in relation to the averaged measurement.

Analysis unit 100 according to FIG. 1 may be provided as a separate system in a separate control unit. Alternatively, this analysis unit 100 may also be housed directly in the gas sensor, however, so that the gas sensor directly generates a first signal, which indicates a hazardous situation because of a leak, and a second signal, which indicates a comfort situation, and optionally outputs them to a further control unit to initiate first and/or second measures.

What is claimed is:

1. A method for analyzing the measured variables of a gas sensor, the method comprising:
detecting measured variables of a gas sensor representing the concentration of the gas to be detected;
in a first analysis mode, as a function of a comparison of at least one measured variable and/or a difference between two successive measured variables to a first threshold value, generating a first gas concentration signal; and
in a second analysis mode, as a function of an average value calculation of at least two measured variables, generating a second gas concentration signal.

2. The method of claim 1, wherein in the first analysis mode, as a function of the overshoot of the first threshold value by the one measured variable and/or the difference between two successive measured variables, a safety-critical situation is recognized, and wherein at least one of a first warning signal and a control signal for initiating countermeasures is generated.

3. The method of claim 1, wherein in the second analysis mode, as a function of the second threshold value being exceeded by the second gas concentration signal, a comfort-critical situation is recognized, and wherein at least one of a second warning signal and a control signal for initiating comfort measures is generated.

4. The method of claim 1, wherein the gas sensor includes at least two detector channels, and wherein the measured variables for generating the first gas concentration signal are detected from a first detector channel and those for generating the second gas concentration signal are detected from a second detector channel.

5. The method of claim 4, wherein balancing of the measured variables of the first and the second detector channels is performed using the measured variables of a reference channel of the gas sensor.

6. An analysis unit for a gas sensor, comprising:
an analysis arrangement configured to analyze measured variables of the gas sensor, by performing the following:
detecting measured variables of a gas sensor representing the concentration of the gas to be detected;
in a first analysis mode, as a function of a comparison of at least one measured variable and/or a difference between two successive measured variables to a first threshold value, generating a first gas concentration signal; and
in a second analysis mode, as a function of an average value calculation of at least two measured variables, generating a second gas concentration signal.

7. The analysis unit of claim 6, wherein the analysis arrangement recognizes a safety-critical situation in the first analysis mode as a function of the first threshold value being exceeded by the one measured variable and/or the difference of two successive measured variables, and wherein the analysis arrangement generates at least one of a first warning signal and a control signal for initiating countermeasures.

8. The analysis unit of claim 6, wherein the analysis arrangement, in the second analysis mode, as a function of a second threshold value being exceeded by the second gas concentration signal, recognizes a comfort-critical situation, and wherein the analysis arrangement generates at least one of a second warning signal and a control signal for initiating comfort measures.

9. The analysis unit of claim 6, wherein the analysis arrangement detects the measured variables for generating the first gas concentration from a first detector channel of the gas sensor and detects those for generating the second gas concentration signal from a second detector channel of the gas sensor.

10. The analysis unit of claim 4, wherein the analysis arrangement balances the measured variables of at least one of the first channel and the second detector channel using the measured variables of a reference channel of the gas sensor.

11. A gas sensor, comprising:
an analysis unit for a gas sensor, including an analysis arrangement configured to analyze measured variables of the gas sensor, by performing the following:
detecting measured variables of a gas sensor representing the concentration of the gas to be detected;
in a first analysis mode, as a function of a comparison of at least one measured variable and/or a difference between two successive measured variables to a first threshold value, generating a first gas concentration signal; and
in a second analysis mode, as a function of an average value calculation of at least two measured variables, generating a second gas concentration signal.

12. The gas sensor of claim 11, wherein the gas sensor has at least two detector channels, in which measured variables are generated independently of one another, which represent the concentration of the gas to be detected.

13. The gas sensor of claim 12, wherein the gas sensor has a reference channel, with the aid of which measured variables are generated independently of the concentration of the gas to be detected.

14. The gas sensor of claim 12, wherein the gas sensor includes a CO₂ gas sensor.

15. The gas sensor of claim 12, wherein the gas sensor includes a CO₂ gas sensor, and the gas detected includes CO₂.

16. The method of claim 1, wherein the gas sensor includes a CO₂ gas sensor.

17. The method of claim 1, wherein the gas sensor includes a CO₂ gas sensor, and the gas detected includes CO₂.

18. The analysis unit of claim 6, wherein the gas sensor includes a CO₂ gas sensor.

19. The analysis unit of claim 6, wherein the gas sensor includes a CO₂ gas sensor, and the gas detected includes CO₂.
